(19) United States
(12) Patent Application Publication
(10) Pub. No.: US 2008/0242998 A1
(43) Pub. Date: Oct. 2, 2008

(54) ULTRASONIC CONTRAST IMAGING METHOD AND ULTRASONIC DIAGNOSTIC APPARATUS

(76) Inventor: Yoichi Suzuki, Tokyo (JP)

Correspondence Address: Patrick W. Rasche;Armstrong Teasdale LLP, Suite 2600, One Metropolitan Square, St. Louis, MO, 63102, US

(21) Appl. No.: 12/055,822

(22) Filed: Mar. 26, 2008

(30) Foreign Application Priority Data
Mar. 30, 2007 (JP) 2007-090443

(51) Int. Cl. A61B 8/00

(52) U.S. Cl. 600/458

(57) ABSTRACT

An ultrasonic contrast imaging method includes: a contrast medium destroying step of transmitting an ultrasonic wave for destroying a contrast medium in a portion close to an ultrasonic probe; and an imaging step of acquiring an ultrasonic image after destruction of the contrast medium in the portion close to the ultrasonic probe.

### BACKGROUND OF THE INVENTION

The present invention relates to an ultrasonic contrast imaging method and an ultrasonic diagnostic apparatus and, more particularly, to an ultrasonic contrast imaging method and an ultrasonic diagnostic apparatus capable of acquiring an ultrasonic image after eliminating the adverse influence of a contrast medium in a portion close to an ultrasonic probe.

In recent years, novel contrast media are being developed. For example, Sonazoid (trademark of Daiichi Sankyo Company, Limited) is well taken in Kupffer cells which do not exist in cancer cells and reflects an ultrasonic wave. It has an advantage that an ultrasonic image having a clear contrast with a cancer part can be captured (see Nikkei Business Daily of Dec. 4, 2006).

However, when a contrast medium excessively exists in a shallow portion (portion close to an ultrasonic probe), an ultrasonic beam is reflected/attenuated by the contrast medium in the shallow portion, and a problem occurs that observation of a deep portion (portion far from the ultrasonic probe) is disturbed.

### SUMMARY OF THE INVENTION

It is desirable that the problem described previously is solved.

In a first aspect, the invention provides an ultrasonic contrast imaging method including: a contrast medium destroying step of transmitting an ultrasonic wave for destroying a contrast medium in a portion close to an ultrasonic probe; and an imaging step of acquiring an ultrasonic image after destruction of the contrast medium in the portion close to the ultrasonic probe.

In the ultrasonic contrast imaging method according to the first aspect, by transmitting an ultrasonic wave, an excessive contrast medium in a portion close to an ultrasonic probe is destroyed. After that, an ultrasonic image is acquired. Consequently, after the adverse influence of the excessive contrast medium in the portion close to the ultrasonic probe is eliminated, an ultrasonic image can be acquired.

In a second aspect of the invention, in the ultrasonic contrast imaging method according to the first aspect, in the contrast medium destroying step, a scan with an ultrasonic beam is continued until predetermined destruction time.

In the ultrasonic contrast imaging method according to the second aspect, by continuing the scan with the ultrasonic beam until the predetermined destruction time (time required to destroy the contrast medium in the portion close to the ultrasonic probe), the excessive contrast medium in the portion close to the ultrasonic probe is destroyed.

In a third aspect of the invention, in the ultrasonic contrast imaging method according to the second aspect, in the contrast medium destroying step, an ultrasonic image is generated and the destruction time is determined on the basis of pixel values in part or all of the ultrasonic image.

In the ultrasonic contrast imaging method according to the third aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of pixel values in part or all of the ultrasonic image. The time required for the determination is set as destruction time.

In a fourth aspect of the invention, in the ultrasonic contrast imaging method according to the third aspect, the destruction time is determined on the basis of an average value of pixel values.

In the ultrasonic contrast imaging method according to the fourth aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of an average value of pixel values in part or all of the ultrasonic image (the average value changes after destruction of the contrast medium). The time required for the determination of destruction is set as destruction time.

In a fifth aspect of the invention, in the ultrasonic contrast imaging method according to the third aspect, the destruction time is determined on the basis of histogram of pixel values.

In the ultrasonic contrast imaging method according to the fifth aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of histogram of pixel values in part or all of the ultrasonic image (the histogram changes after destruction of the contrast medium). The time required for the determination of destruction is set as destruction time.

In a sixth aspect of the invention, in the ultrasonic contrast imaging method according to the second aspect, in the contrast medium destroying step, ultrasonic images are generated, and the destruction time is determined on the basis of frame correlation between the ultrasonic images.

In the ultrasonic contrast imaging method according to the sixth aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of frame correlation of ultrasonic images sequentially acquired (the frame correlation decreases after destruction of the contrast medium). The time required for the determination of destruction is set as destruction time.

In a seventh aspect of the invention, in the ultrasonic contrast imaging method according to any of the second to sixth aspect, in the contrast medium destroying step, sound pressure of the ultrasonic beam is changed during the scan.

In the ultrasonic contrast imaging method according to the seventh aspect, by changing the sound pressure of the ultrasonic beam, the excessive contrast medium in the portion close to the ultrasonic probe can be destroyed under proper control.

In an eighth aspect of the invention, in the ultrasonic contrast imaging method according to any of the second to seventh aspects, in the contrast medium destroying step, focus of the ultrasonic beam is moved during the scan.

In the ultrasonic contrast imaging method according to the eighth aspect, by changing the focus of the ultrasonic beam, the excessive contrast medium in the portion close to the ultrasonic probe can be destroyed under proper control.

In a ninth aspect, the invention provides an ultrasonic diagnostic apparatus including: a contrast medium destruction ultrasonic wave transmitting device for transmitting an ultrasonic wave for destroying a contrast medium in a portion close to an ultrasonic probe; and an imaging device for acquiring an ultrasonic image after destruction of the contrast medium in the portion close to the ultrasonic probe.

In the ultrasonic diagnostic apparatus according to the ninth aspect, an ultrasonic wave is transmitted to destroy the excessive contrast medium in the portion close to the ultrasonic probe. After that, an ultrasonic image is acquired. Consequently, after eliminating the adverse influence of the excessive contrast medium in the portion close to the ultrasonic probe, an ultrasonic image can be acquired.

In a tenth aspect of the invention, in the ultrasonic diagnostic apparatus according to the ninth aspect, in the contrast medium destruction ultrasonic wave transmitting device, a scan with an ultrasonic beam is continued until predetermined destruction time.

In the ultrasonic diagnostic apparatus according to the tenth aspect, by continuing a scan with an ultrasonic beam until predetermined destruction time (time required to destroy the contrast medium in the portion close to the ultrasonic probe), the excessive contrast medium in the portion close to the ultrasonic probe is destroyed.

In an eleventh aspect of the invention, in the ultrasonic diagnostic apparatus according to the tenth aspect, in the contrast medium destruction ultrasonic wave transmitting device, an ultrasonic image is generated, and the destruction time is determined on the basis of pixel values in part or all of the ultrasonic image.

In the ultrasonic diagnostic apparatus according to the eleventh aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of pixel values in part or all of the ultrasonic image. The time required for the determination is set as destruction time.

In a twelfth aspect of the invention, in the ultrasonic diagnostic apparatus according to the eleventh aspect, the destruction time is determined on the basis of an average value of pixel values.

In the ultrasonic diagnostic apparatus according to the twelfth aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of an average value of pixel values in part or all of the ultrasonic image (the average value changes after destruction of the contrast medium). The time required for the determination of destruction is set as destruction time.

In a thirteenth aspect of the invention, in the ultrasonic diagnostic apparatus according to the eleventh aspect, the destruction time is determined on the basis of histogram of pixel values.

In the ultrasonic diagnostic apparatus according to the thirteenth aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of histogram of pixel values in part or all of the ultrasonic image (the histogram changes after destruction of the contrast medium). The time required for the determination of destruction is set as destruction time.

In a fourteenth aspect of the invention, in the ultrasonic diagnostic apparatus according to the tenth aspect, in the contrast medium destruction ultrasonic wave transmitting device, ultrasonic images are generated, and the destruction time is determined on the basis of frame correlation between the ultrasonic images.

In the ultrasonic diagnostic apparatus according to the fourteenth aspect, while performing a scan with an ultrasonic beam to destroy the excessive contrast medium in the portion close to the ultrasonic probe, an ultrasonic image is generated. Whether the excessive contrast medium in the portion close to the ultrasonic probe has been destroyed or not is determined on the basis of frame correlation of ultrasonic images sequentially acquired (the frame correlation decreases after destruction of the contrast medium). The time required for the determination of destruction is set as destruction time.

In a fifteenth aspect of the invention, in the ultrasonic diagnostic apparatus according to any of the tenth to fourteenth aspects, in the contrast medium destruction ultrasonic wave transmitting device, sound pressure of the ultrasonic beam is changed during the scan.

In the ultrasonic diagnostic apparatus according to the fifteenth aspect, by changing the sound pressure of the ultrasonic beam, the excessive contrast medium in the portion close to the ultrasonic probe can be destroyed under proper control.

In a sixteenth aspect of the invention, in the ultrasonic diagnostic apparatus according to any of the tenth to fifteenth aspects, in the contrast medium destruction ultrasonic wave transmitting device, focus of the ultrasonic beam is moved during the scan.

In the ultrasonic diagnostic apparatus according to the sixteenth aspect, by changing the focus of the ultrasonic beam, the excessive contrast medium in the portion close to the ultrasonic probe can be destroyed under proper control.

In the ultrasonic contrast imaging method and the ultrasonic diagnostic apparatus of the invention, after destroying the excessive contrast medium existing in the portion close to the ultrasonic probe, an ultrasonic image is captured. Thus, after eliminating the adverse influence of the excessive contrast medium in the portion close to the ultrasonic probe, an ultrasonic image can be acquired.

The ultrasonic diagnostic apparatus of the invention can be used for ultrasonic imaging using a novel contrast medium.

Further objects and advantages of the present invention will be apparent from the following description of the preferred embodiments of the invention as illustrated in the accompanying drawings.

### BRIEF DESCRIPTION OF DRAWINGS

FIG. 1 is a diagram illustrating the configuration of an ultrasonic diagnostic apparatus of a first embodiment.

FIG. 2 is a flowchart showing the procedure of contrast imaging process in the first embodiment.

FIG. 3 is a conceptual diagram showing an ultrasonic image in a state where a contrast medium excessively exists in a shallow portion.

FIG. 4 is a conceptual diagram showing an ultrasonic image in a state where the contrast medium existing in the shallow portion is destructed to some extent.

FIG. 5 is a conceptual diagram showing an ultrasonic image in a state where the contrast medium existing in the shallow portion is destructed fully.

### DETAILED DESCRIPTION OF THE INVENTION

The invention will be described more specifically hereinbelow by an embodiment shown in the diagrams. The invention, however, is not limited by the embodiments.

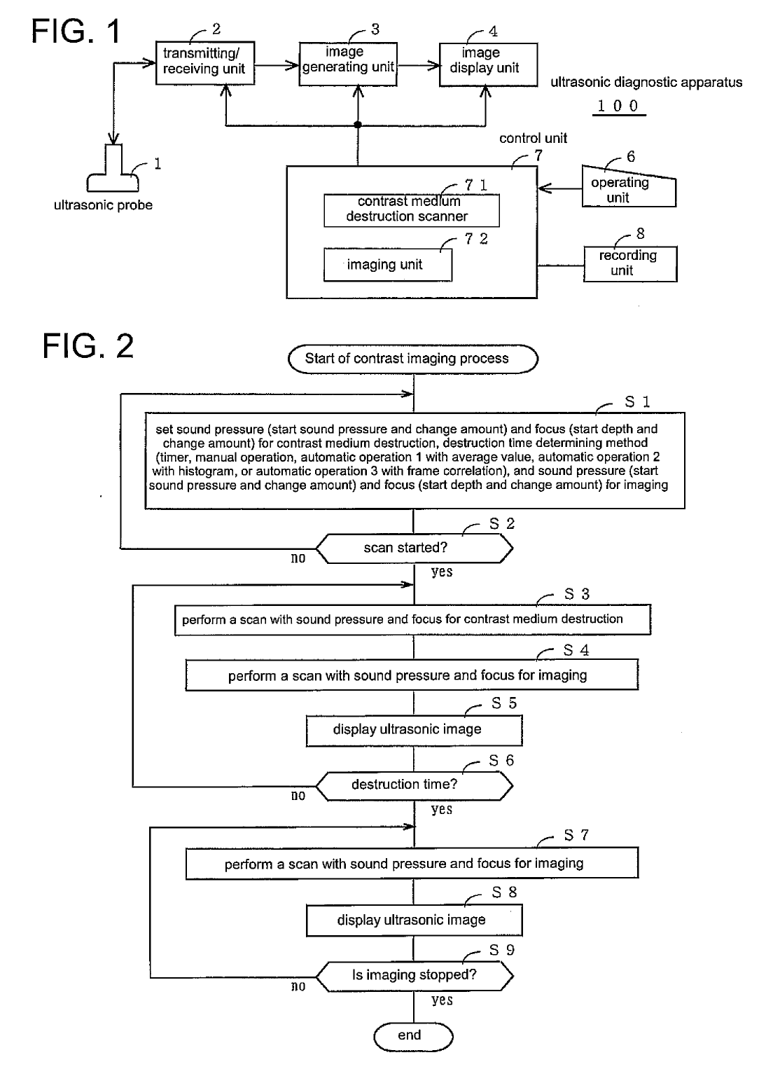

First Embodiment. FIG. 1 is a diagram illustrating the configuration of an ultrasonic diagnostic apparatus 100 according to a first embodiment.

The ultrasonic diagnostic apparatus 100 has an ultrasonic probe 1, a transmitting/receiving unit 2 for driving the ultrasonic probe 1 to scan a subject with an ultrasonic beam, an image generating unit 3 for generating an ultrasonic image on the basis of the obtained reception data, an image display unit 4 for displaying an ultrasonic image or the like, an operating unit 6 operated by the operator to give an instruction and data, a control unit 7 for controlling general operation, and a recording unit 8 for recording an ultrasonic image or the like.

The control unit 7 includes a contrast medium destruction scanner 71 for transmitting an ultrasonic wave to destroy a contrast medium in a portion close to the ultrasonic probe 1, and an imaging unit 72 for capturing an ultrasonic image after destruction of the contrast medium in the portion close to the ultrasonic probe 1.

FIG. 2 is a flowchart showing the contrast imaging process performed by the ultrasonic diagnostic apparatus 100.

In step S1, the operator sets a sound pressure and focus for contrast medium destruction, a destruction time determining method, and a sound pressure and focus for imaging.

As the sound pressure for contrast medium destruction, “start sound pressure” and “change amount” can be set. When the “change amount” is set to “0”, scan is repeated at the “start sound pressure”. When the “change amount” is set to a “positive value”, the sound pressure gradually increases from the “start sound pressure” by “change amount” scan by scan. When the “change amount” is set to a “negative value”, the sound pressure gradually decreases from the “start sound pressure” by “change amount” scan by scan.

As the focus for contrast medium destruction, “start depth” and “change amount” can be set. When the “change amount” is set to “0”, the scan is repeated with the focus at the “start depth”. When the “change amount” is set to “negative value”, the focus becomes shallower by “change amount” scan by scan from the “start depth”.

As the destruction time determining method, any of “timer”, “manual operation”, “automatic operation 1: average value”, “automatic operation 2: histogram”, and “automatic operation 3: frame correlation” can be selected.

When “timer” is selected, “timeup time” can be set. The “timeup time” corresponds to destruction time.

When “manual operation” is selected, the time until time point when a stop instruction is given by the operator corresponds to destruction time.

When “automatic operation 1: average value” is selected, after the scan for contrast medium destruction, scan for imaging is performed, and an ultrasonic image is generated. When the average pixel value of the ultrasonic image exceeds a threshold, conceptually, time of a change from a dark image to a light image corresponds to destruction time.

When “automatic operation 2: histogram” is selected, after the scan for contrast medium destruction, scan for imaging is performed, and an ultrasonic image is generated. Time until a change in the predetermined pattern of the histogram of the ultrasonic image, conceptually, a change in a pattern having many dark pixels to a pattern having many light pixels corresponds to destruction time.

When “automatic operation 3: frame correlation” is selected, after the scan for contrast medium destruction, scan for imaging is performed, and an ultrasonic image is generated. When the correlation value between an ultrasonic image of a preceding frame and an ultrasonic image of the present frame becomes smaller than the threshold and, after that, becomes larger than the threshold, conceptually, time since a change between the ultrasonic image of the preceding frame and an ultrasonic image of the present frame increases and then decreases corresponds to destruction time.

As the sound pressure for imaging, “start sound pressure” and “change amount” can be set. When the “change amount” is set to “0”, scan is repeated at the “start sound pressure”. When the “change amount” is set to a “positive value”, the sound pressure gradually increases from the “start sound pressure” by “change amount” scan by scan. When the “change amount” is set to a “negative value”, the sound pressure gradually decreases from the “start sound pressure” by “change amount” scan by scan.

As the focus for imaging, “start depth” and “change amount” can be set. When the “change amount” is set to “0”, the scan is repeated with the focus at the “start depth”. When the “change amount” is set to “negative value”, the focus becomes shallower by “change amount” scan by scan from the “start depth”.

Step S1 is repeated until the operator instructs start of a scan in step S2. When the operator instructs start of a scan, the apparatus advances to step S3.

In step S3, a scan is performed only by one frame with the sound pressure and focus for contrast medium destruction.

In step S4, a scan is performed only by one frame with the sound pressure and focus for imaging.

In step S5, an ultrasonic image is displayed.

Steps S3 to S5 are repeated until destruction time is detected in step S6. When the destruction time arrives, the apparatus advances to step S7.

In step S7, a scan is performed only by one frame with the sound pressure and focus for imaging, and an ultrasonic image is generated.

In step S8, the ultrasonic image is displayed.

Steps S7 and S8 are repeated until the operator instructs to stop the imaging in step S9. When the operator instructs to stop the imaging, the process is finished.

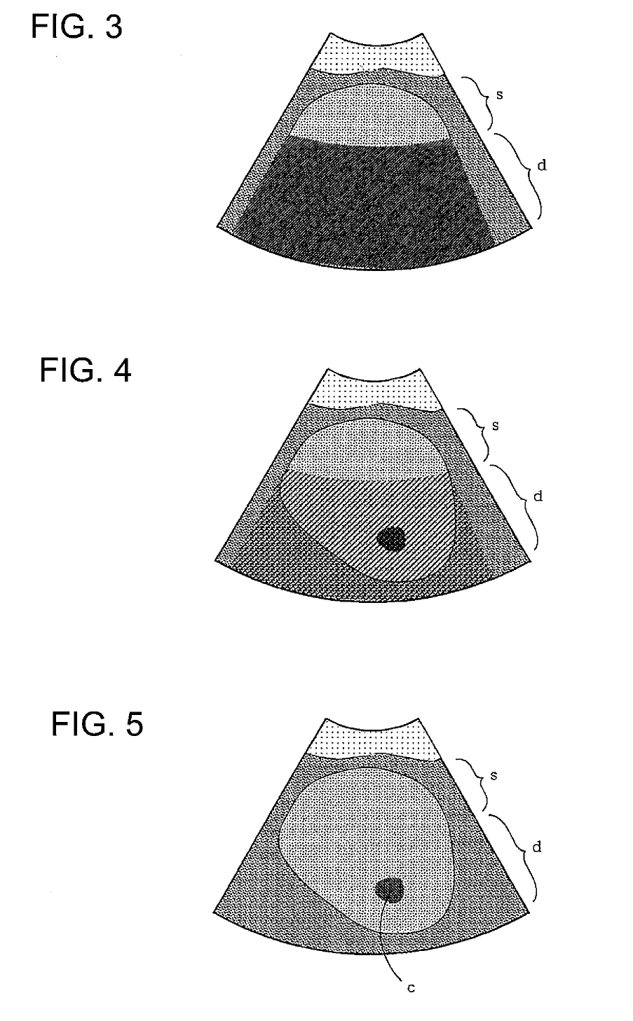

FIG. 3 is a conceptual diagram showing an ultrasonic image in a state where a contrast medium excessively exists in a shallow portion “s”.

The ultrasonic beam is reflected/attenuated by the contrast medium in the shallow portion “s” and observation of a deep portion “d” is disturbed.

FIG. 4 is a conceptual diagram showing an ultrasonic image in a state where the contrast medium existing in the shallow portion “s” is destructed to some extent.

FIG. 5 is a conceptual diagram showing an ultrasonic image in a state where the contrast medium existing in the shallow portion “s” is destructed fully.

Observation can be made excellently to the deep portion “d”, and a cancer “c” can be observed.

In the ultrasonic diagnostic apparatus 100 of the first embodiment, after destruction of an excessive contrast medium existing in a portion near the ultrasonic probe 1, an ultrasonic image is acquired. Consequently, after the adverse influence of the excessive contrast medium in the portion near the ultrasonic probe 1 is eliminated, an ultrasonic image can be obtained.

Many widely different embodiments of the invention may be configured without departing from the spirit and the scope of the present invention. It should be understood that the present invention is not limited to the specific embodiments described in the specification, except as defined in the appended claims.

1. An ultrasonic contrast imaging method comprising:
destroying a contrast medium by transmitting an ultrasonic wave for destroying the contrast medium in a portion close to an ultrasonic probe; and
acquiring an ultrasonic image after destruction of the contrast medium in the portion close to the ultrasonic probe.

2. The ultrasonic contrast imaging method according to claim 1, wherein destroying a contrast medium further comprises continuing a scan with an ultrasonic beam until a predetermined destruction time.

3. The ultrasonic contrast imaging method according to claim 2, wherein destroying a contrast medium further comprises generating an ultrasonic image and determining the destruction time based on one of pixel values in a portion of the ultrasonic image and all pixel values of the ultrasonic image.

4. The ultrasonic contrast imaging method according to claim 3, wherein determining the destruction time further comprises determining the destruction time based on one of an average value of pixel values in a portion of the ultrasonic image and an average value of all pixel values of the ultrasonic image.

5. The ultrasonic contrast imaging method according to claim 3, wherein determining the destruction time further comprises determining the destruction time based on one of a histogram of pixel values in a portion of the ultrasonic image and a histogram of all pixel values of the ultrasonic image.

6. The ultrasonic contrast imaging method according to claim 2, wherein destroying the contrast medium further comprises generating ultrasonic images and determining the destruction time based on a frame correlation between the ultrasonic images.

7. The ultrasonic contrast imaging method according to claim 2, wherein destroying the contrast medium further comprises changing a sound pressure of ultrasonic beam during the scan.

8. The ultrasonic contrast imaging method according to claim 2, wherein destroying the contrast medium further comprises moving a focus of an ultrasonic beam during the scan.

9. An ultrasonic diagnostic apparatus comprising:
a contrast medium destruction ultrasonic wave transmitting device for transmitting an ultrasonic wave for destroying a contrast medium in a portion close to an ultrasonic probe; and
an imaging device for acquiring an ultrasonic image after destruction of the contrast medium in the portion close to the ultrasonic probe.

10. The ultrasonic diagnostic apparatus according to claim 9, wherein the contrast medium destruction ultrasonic wave transmitting continues a scan with an ultrasonic beam until a predetermined destruction time.

11. The ultrasonic diagnostic apparatus according to claim 10, wherein the contrast medium destruction ultrasonic wave transmitting generates an ultrasonic image and determines the destruction time based on one of pixel values in a portion of the ultrasonic image and all pixel values of the ultrasonic image.

12. The ultrasonic diagnostic apparatus according to claim 11, wherein the destruction time is determined based on one of an average value of pixel values of a portion of the ultrasonic image and an average value of all pixel values of the ultrasonic image.

13. The ultrasonic diagnostic apparatus according to claim 11, wherein the destruction time is determined based on one of a histogram of pixel values of a portion of the ultrasonic image and a histogram of all pixel values of the ultrasonic image.

14. The ultrasonic diagnostic apparatus according to claim 10, wherein the contrast medium destruction ultrasonic wave transmitting device generates a plurality of ultrasonic images and determines the destruction time based on a frame correlation between the ultrasonic images.

15. The ultrasonic diagnostic apparatus according to claim 10, wherein the contrast medium destruction ultrasonic wave transmitting device changes a sound pressure of an ultrasonic beam during the scan.

16. The ultrasonic diagnostic apparatus according to claim 11, wherein the contrast medium destruction ultrasonic wave transmitting device changes a sound pressure of an ultrasonic beam during the scan.

17. The ultrasonic diagnostic apparatus according to claim 12, wherein the contrast medium destruction ultrasonic wave transmitting device changes a sound pressure of an ultrasonic beam during the scan.

18. The ultrasonic diagnostic apparatus according to claim 10, wherein the contrast medium destruction ultrasonic wave transmitting device moves a focus of an ultrasonic beam during the scan.

19. The ultrasonic diagnostic apparatus according to claim 11, wherein the contrast medium destruction ultrasonic wave transmitting device moves a focus of an ultrasonic beam during the scan.

20. The ultrasonic diagnostic apparatus according to claim 12, wherein the contrast medium destruction ultrasonic wave transmitting device moves a focus of an ultrasonic beam during the scan.
